(19) United States
(12) Patent Application Publication
(10) Pub. No.: US 2026/0256740 A1
(43) Pub. Date: Sep. 3, 2026

(54) PHARMACEUTICAL COMPOSITION COMPRISING CYCLOHEX-2-ENYLIDENE / 3,3-DIMETHYLINDOLINE COMPOUNDS; METHODS OF TREATING HUMAN DISEASES; AND METHODS OF INDUCING A CALCIUM SENSITIZING EFFECT IN A SUBJECT

(71) Applicant: Curadel Surgical Innovations, Inc., Natick, MA (US)

(72) Inventor: John V. Frangioni, Weston, MA (US)

(21) Appl. No.: 18/870,873

(22) PCT Filed: Jun. 1, 2023
(86) PCT No.: PCT/US2023/024169

§ 371 Date: Dec. 2, 2024

Related U.S. Application Data
(60) Provisional application No. 63/348,126, filed on Jun. 2, 2022.
(60) Provisional application No. 63/397,286, filed on Aug. 11, 2022.

(51) Int. Cl. A61K 31/4045

(52) U.S. Cl. CPC A61K 31/4045

(57) ABSTRACT

This disclosure relates to novel pharmaceutical compositions comprising cyclohex-2-enylidene/3, 3-dimethylindoline compounds; a method of treating or ameliorating a cardiovascular disease or disorder, asthma, or narcolepsy in a subject compromising the administration of a one or more compounds or compositions comprising the cyclohex-2-enylidene/3, 3-dimethylindoline compounds; a method of inducing a calcium sensitizing effect in a myocardial cell or a subject compromising the administration of a one or more compounds or compositions comprising the cyclohex-2-enylidene/3, 3-dimethylindoline compounds; and kits for administration of the methods.

### CROSS-REFERENCE TO RELATED APPLICATIONS

This application claims priority to U.S. Provisional Patent Application Ser. No. 63/348,126 filed Jun. 2, 2022 and from U.S. Provisional Patent Application Ser. No. 63/397,286 filed Aug. 11, 2022 disclosure of which are both incorporated herein by reference.

### STATEMENT REGARDING FEDERALLY SPONSORED RESEARCH OR DEVELOPMENT

This disclosure was made with Government support under project number R01-CA-207500 by the National Institutes of Health. The government has certain rights in the disclosure.

### BACKGROUND

Isoproterenol, or isoprenaline, is a beta-1 and beta-2 adrenergic receptor agonist used for the treatment of bradycardia, heart block, asthma, bronchospasm during anesthesia, hypovolemic shock, septic shock, low cardiac output (hypoperfusion) states, congestive heart failure, and cardiogenic shock. It is a non-selective β adrenoceptor agonist that is the isopropylamine analog of epinephrine (adrenaline). The administration and subsequent post-administration monitoring of this medication are complex and necessitate an interprofessional approach to its usage. Adverse effects of Isoproterenol include nervousness, headache, dizziness, nausea, visual blurring, tachycardia, palpitations, angina, Adams-Stokes attacks, pulmonary edema, hypertension, hypotension, ventricular arrhythmias, tachyarrhythmias, difficulty breathing, sweating, mild tremors, weakness, flushing, and pallor. Isoproterenol has also been reported to cause insulin resistance leading to diabetic ketoacidosis.

In addition, calcium sensitizers are a class of inotropic compounds which act to increase myocardial contractility in patients with cardiac failure. Other classes of drugs for this indication include catecholamines and phosphodiesterase-III (PDE) inhibitors. However, improving myocardial performance with catecholamines and PDE inhibitors leads to increased intracellular calcium concentration as an unavoidable side effect. Such an increase results in an increase in incidences of harmful or fatal arrhythmias. Calcium sensitizers improve myocardial performance by acting on contractile proteins without increasing intracellular calcium load. Calcium sensitizers enhance myocardial performance without increasing myocardial oxygen consumption and without provoking fatal arrhythmias.

To date, only a few calcium sensitizers have been studied for the treatment of cardiac failure. Nevertheless, these compounds have generally resulted in PDE inhibitory properties with arrhythmogenic risks. In addition, the known calcium sensitizers are lipophilic and must traverse the cell to bind to intracellular targets.

As such, there remains a need for new and improved treatments for cardiovascular diseases which are have increased potency and are more easily tolerated by patients. In particular, there is a current need for new and improved calcium sensitizers which act in novel ways to avoid the downfalls of previous calcium sensitizers.

### SUMMARY

PanLN and PanLN-forte have been used in recent years as near infrared (NIR) fluorescence contrast imaging agents. For example, in WO2015066290A1/US20160263249A1/U.S. Pat. No. 11,077,210B2 these compounds are identified as imaging agents for lymph nodes and sentinel lymph nodes (identified as MM25 and A64, respectively).

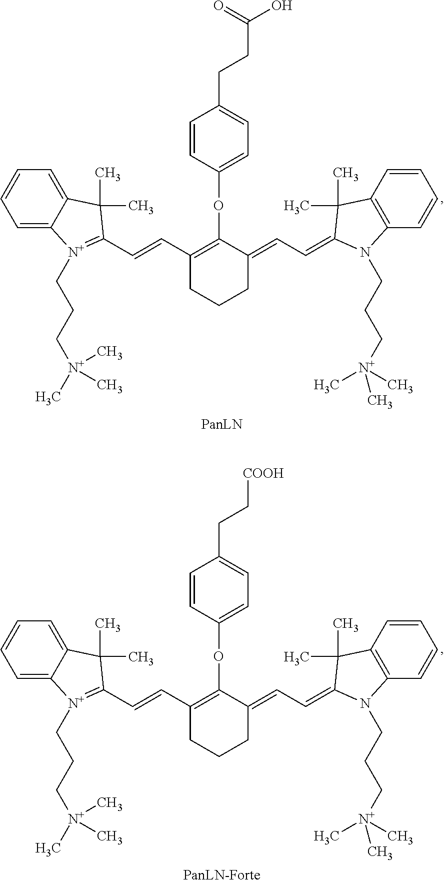 [SMILES: CC1(C)C(/C=C/C2=C(Oc3ccc(CCC(=O)O)cc3)C(=C/C=C3/N(CCC[N+](C)(C)C)c4ccccc4C3(C)C)/CCC2)=[N+](CCC[N+](C)(C)C)c2ccccc21 ; CC1(C)C(/C=C/C2=C(Oc3ccc(CCC(=O)O)cc3)C(=C/C=C3/N(CCC[N+](C)(C)C)c4ccccc4C3(C)C)/CCC2)=[N+](CCC[N+](C)(C)C)c2ccccc21]

This disclosure provides for a method of using these compounds as a pharmaceutical composition for the treatment of a variety of diseases, including, but not limited to, cardiovascular diseases, asthma, and narcolepsy. This disclosure also provides methods of treating heart disease, asthma, and narcolepsy as well as kits for the treatment of these diseases.

In one aspect, the disclosure provides a pharmaceutical composition comprising 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof and a pharmaceutically acceptable excipient or carrier. This compound is also referred to as PanLN-Forte or A64.

In one aspect, the disclosure provides a pharmaceutical composition comprising 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof and a pharmaceutically acceptable excipient or carrier. This compound is also referred to as PanLN or MM25.

In another aspect, the disclosure provides a method of treating a cardiovascular disease, asthma, or narcolepsy in a patient in need thereof comprising administering a pharmaceutically effective amount of 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof.

In certain embodiments, the cardiovascular disease of the method of the disclosure is heart failure, heart disease, heart block, cardiac arrest from heart block, bradycardia, cardiogenic shock, hypovolemic shock, Torsades de pointes, ventricular arrhythmia, Short QT syndrome, or electrical storms associated with Brugada syndrome.

In some embodiments of the method of the disclosure, the compound is administered in the form of a pharmaceutical composition wherein the pharmaceutical composition comprises the compound and one or more pharmaceutically acceptable excipients or carriers.

In another aspect, the disclosure provides a patient care kit, comprising:
(a) a unit dosage form or a bulk-dosage form of 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof; and
(b) instructions for administration to a subject having a cardiovascular disease, asthma, or narcolepsy.

In another aspect, the disclosure provides a method of calcium sensitization in a myocardial cell comprising contacting a myocardial cell with 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce calcium sensitization in the cell.

In yet another aspect, the disclosure provides a method of calcium sensitization in a subject in need thereof comprising administering 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce calcium sensitization in the subject.

In yet another aspect, the disclosure provides a method of treating a heart condition in a subject in need thereof comprising administering 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce calcium sensitization in the subject. In certain embodiments, the heart condition is heart failure; cardiogenic shock; septic shock; pulmonary hypertension; bradycardia; high blood pressure, angina, arrhythmia hypertrophic obstructive cardiomyopathy and allergic reactions. In other embodiments, the subject is a human.

### BRIEF DESCRIPTIONS OF THE DRAWINGS

FIG. 1 shows a plot and table depicting the percent changes in contractility amplitude for PanLN Triflutate at 1 Hz. (n=12)

FIG. 2 shows a plot and table depicting the incidence in AC, CF, Alternans and STV for PanLN Triflutate. (n=12)

FIG. 3 shows the typical contractility transients recorded from a ventricular cardiomyocyte at a pacing of 1 Hz in the presence of vehicle control and after exposure to 1 μM PanLN Triflutate.

FIG. 4 shows the typical contractility transients recorded from a ventricular cardiomyocyte at a pacing of 1 Hz in the presence of vehicle control and after exposure to 5 μM PanLN Triflutate.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity

A/an: The articles “a” and “an” as used herein mean one or more when applied to any feature in embodiments and implementations of this disclosure described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data. All numerical values within the detailed description and the claims herein are modified by “about” or “approximately” the indicated value, and take into account experimental error and variations that would be expected by a person having ordinary skill in the art.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Any device or method or system described herein can be comprised of, can consist of, or can consist essentially of any one or more of the described elements.

In particular, as used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

Ranges: Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds). In the figures, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

As used herein, the term “administration” or “administering” of the subject compound refers to providing a compound of the disclosure and/or prodrugs thereof to a subject in need of diagnosis or treatment.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

As used herein, the term “effective” is used to describe an amount of a compound, composition or component which, when used within the context of its intended use, effects an intended result. The term effective subsumes all other effective amount or effective concentration terms, which are otherwise described or used in the present application.

Other definitions appear in context throughout the disclosure.

### Compounds and Synthesis

It has been found that certain compounds are for the treatment of diseases in patients. In particular, it has been found that PanLN and PanLN-forte are particularly effective for the treatment of cardiovascular diseases, asthma, and narcolepsy.

In one aspect, the disclosure provides a pharmaceutical composition comprising 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof. This compound is also referred to as PanLN-Forte or A64.

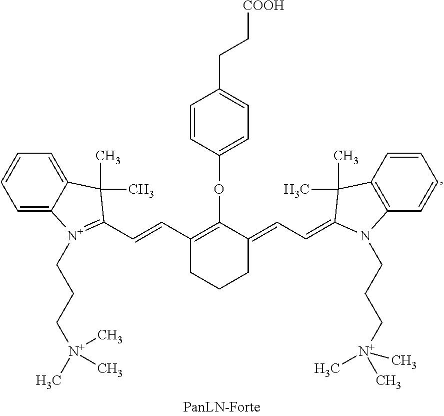 [SMILES: CC1(C)C(/C=C/C2=C(Oc3ccc(CCC(=O)O)cc3)C(=C/C=C3/N(CCC[N+](C)(C)C)c4ccccc4C3(C)C)/CCC2)=[N+](CCC[N+](C)(C)C)c2ccccc21]

In one aspect, the disclosure provides a pharmaceutical composition comprising 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof. This compound is also referred to as PanLN or MM25.

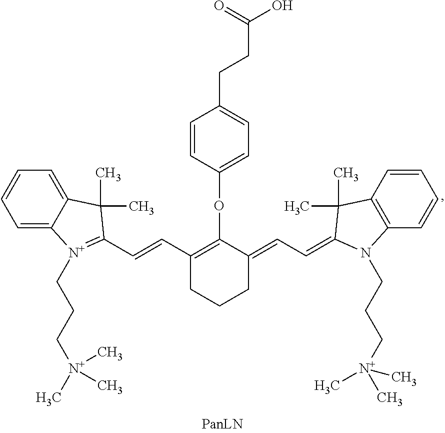 [SMILES: CC1(C)C(/C=C/C2=C(Oc3ccc(CCC(=O)O)cc3)C(=C/C=C3/N(CCC[N+](C)(C)C)c4ccccc4C3(C)C)/CCC2)=[N+](CCC[N+](C)(C)C)c2ccccc21]

These compounds can be prepared using a variety of methods, some of which are known in the art. For example, the compounds can be prepared using conventional methods of synthetic organic chemistry (see, e.g., Michael B. Smith, “March's Advanced Organic Chemistry: Reactions, Mechanisms, and Structure, 7th Edition”, Wiley (2013)).

For example, PanLN-Forte can be synthesized using the syntheses described in schemes 1-3 shown below. General references for the syntheses described in the scheme 1 is found in Henary, M. et al. Bioorg. & Med. Chem. Let. 22, 242-1246, (2012), Henary, M. et al. J. Heterocycl. Chem. 46:84-87, (2009), Henary, M. et al. Dyes and Pigments. 99, 1107-1116 (2013), Henary, M. et al. Heterocycl. Commun. 19 (1), 1-11 (2013), Mojzych, M. et al. Topics in Heterocyclic, Springer-Verlag Berlin Heidelberg. 14, 1-9 (2008), Strekowski, L. et al. J. Org. Chem. 57, 4578-80 (1992), Halder, S. et. al. Eur. J. Med. Chem. 54, 647-59 (2012), Sakiko, A. et al. Chem.-A Eur. J., 15, 9191-9200 (2009), Chang, Y-T. et al. Chem. Commun. 47, 3514-3516 (2011), Myochin, T. J. Am. Chem. Soc. 134, 13730-13737 (2012), Briza, T. et al. Chem. Comm. 16, 1901-1903 (2008), Chang, Y-T. et al. Chem. Commum. 46, 7406-7408 (2010), Zaheer, A., et al. Molecular Imag. 1 (4), 1536-0121 (2002), Misra, P. et al. J Nucl Med. 2007 August; 48(8):1379-89, and Humblet, V. et al, J Med Chem. 2009 Jan. 22; 52(2):544-50.

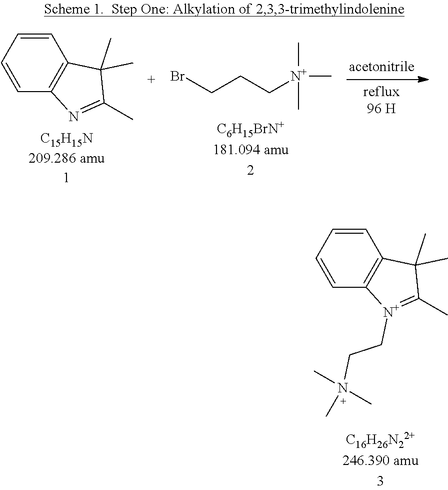

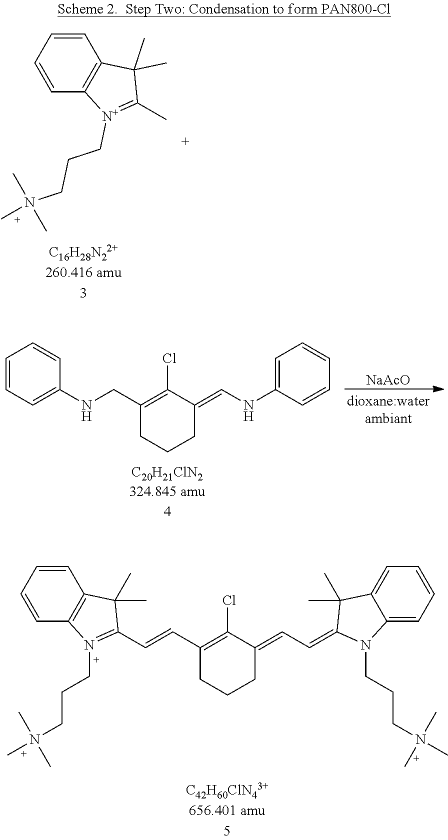

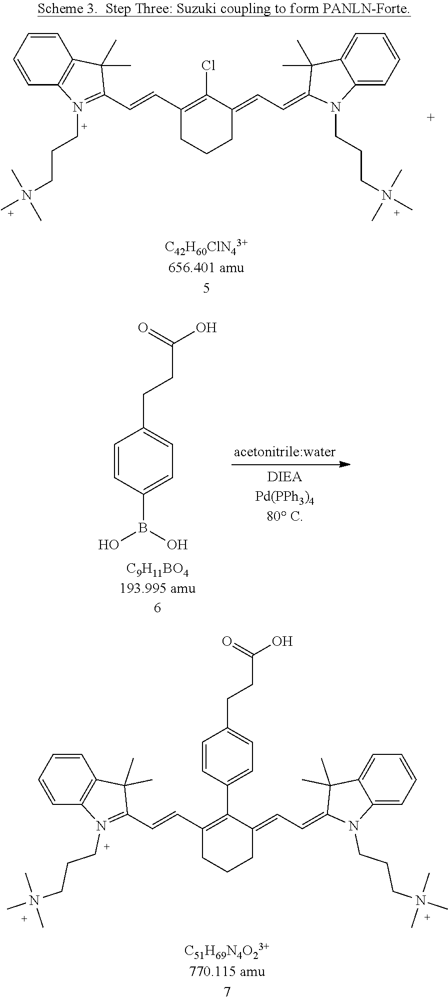

### Step One: Alkylation of 2,3,3-trimethyl-3H-indole

The alkylation of the commercially available 2,3,3-trimethyl-3H-indole with the salt (3-bromopropyl)trimethylammonium bromide is performed at reflux in acetonitrile. The reaction progresses between 1 and 2 percent per hour and requires approximately 96 hours to complete. Upon cooling the reaction mixture, the product will precipitate from the reaction solvent in high purity and yield. The reaction solvent can be removed either by centrifugation or by filtration. The product is washed with fresh acetonitrile.

Target material is lost to the reaction solvent to give an overall yield of 81.0% on a 10 g scale. The target product is collected as a di-bromide salt with a molecular weight of 420.23 g/mol. The structure and molecular weight of the collected bromide salt is shown below.

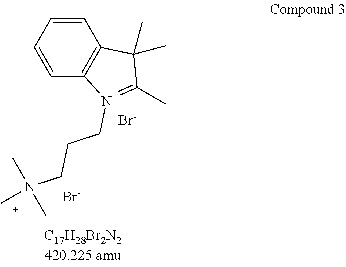

### Step Two: Condensation of quat-3H-indole and Center Core

The condensation of the target molecule made in step 1 and the commercially available center core (4) is performed in a mildly basic environment. The use of dioxane and a small portion of water appears to prevent side-reactions that were previously reported. Previously, this reaction required column purification due to side-reactions occurring. These side reactions were SN1 attacks at the chlorinated carbon by the leaving aniline group on compound 4. This reaction is extremely base sensitive, excess base will result in SN1 attacks.

Target material is lost to the reaction solvent to give an overall yield of 73.5% on a 10 g scale. The target product is collected as a tri-bromide salt with a molecular weight of 896.115 g/mol. The structure and molecular weight of the collected bromide salt is shown below.

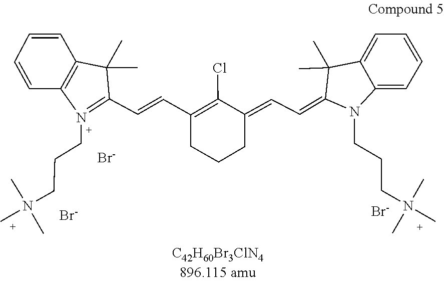

### Step Three: Suzuki-Miyaura Coupling

The Suzuki-Miyaura synthesis to form the target molecule is consists of reacting boronic acid linker to the organohalide using a palladium (0) complex as a catalyst. These reactions require a base to progress, but as with many heptamethine fluorophores, excess base will degrade the final product. In our experience, organic bases such as DIEA are too strong to use if in excess and must be used in equivalent molar amounts. This reaction has been completed successfully using both sodium acetate and N-methylmorpholine.

Target material is lost to the reaction solvent to give an overall yield of 87.2% on a 1 g scale. The target product is collected as a trifluoroacetate salt with a molecular weight of 995.14 g/mol. The structure and molecular weight of the collected bromide salt is shown below.

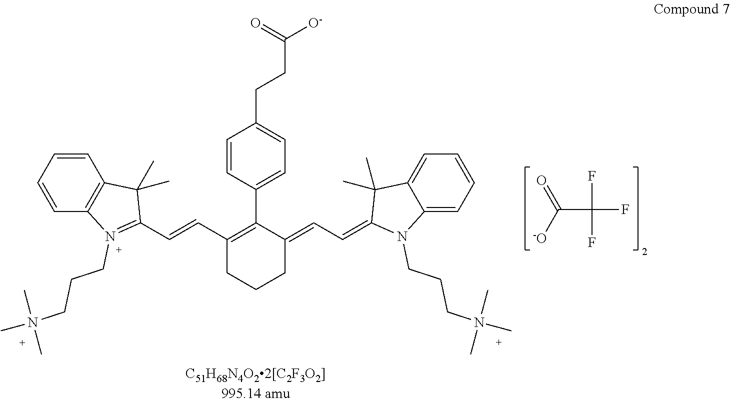

### Step Three: Alternative for PanLN

PanLN can be synthesized in a similar method as PanLN-Forte but by substituting 3-(4-hydroxyphenyl) propanoic acid in place of the 3-(4-boronophenyl) propanoic acid. This reaction does not require the inclusion of a catalyst and can be run, for example, in a mixture of NaH and DMF at 90° C. for approximately 5 hours.

The compounds of the disclosure may also be modified with an acid modifier or other ester or with a transport-inducing cofactor to increase absorption or bioavailability of the compound. The compounds of the disclosure may also be modified with a targeting ligand. The “targeting ligand” is a moiety that binds with some specificity or selectivity to a particular tissue or biological target. The tissue or biological target can include normal tissues as well as abnormal or diseased tissues. Targeting ligands can be selected from specific proteins, protein fragments, peptides, antibodies, carbohydrates, or antigens described, e.g., in Frangioni et al. in “Modified PSMA Ligands and Uses Related Thereto,” WO 02/098885, filed on Feb. 7, 2002 (now issued as U.S. Pat. No. 6,875,886). In particular embodiments, the targeting ligand targets cardiovascular cells, in particular ventricular myocytes.

### Receptor Activity

Without being limited by theory, PanLN and PanLN-forte are believed to not significantly increase intracellular calcium, either from intrinsic or extrinsic pools.

Similarly, NIR cellular microscopy shows that PanLN and PanLN-fort does not appear to cross the plasma membrane of living cells. Without being limited by theory, PanLN and PanLN-forte are believed unlikely to directly bind myofilaments.

Without being limited by theory, PanLN and PanLN-forte are believed to increase contractility of ventricular myocytes. In certain embodiments, the increase in contractility of ventricular myocytes is about 100% of a baseline. In certain other embodiments, the increase in contractility of ventricular myocytes is about 250% of baseline. The increase in contractility of ventricular myocytes is believed to be dose-dependent.

PanLN and PanLN-forte bind to a number of drug receptors. Without being limited by theory, PanLN and PanLN-forte are believed to be effective in treating the diseases and disease states of this disclosure based on binding to one or more of the following receptors: Adrenergic α1D receptors, Adrenergic α2A receptors, Adrenergic α2C receptors, Bradykinin B1 receptors, Inositol Trisphosphate IP3 receptors, Muscarinic M1 receptors, Muscarinic, Oxotremorine M receptors, Orexin OX1 receptors, Serotonin (5-Hydroxytryptamine) 5-HT2C receptors, Tachykinin NK2 receptors, or combinations thereof.

### Calcium Sensitization

Calcium signaling in cardyomyocyte function has been described in Cold Spring Harb Perspect Biol 2020; 12:a035428. In particular, it is believed that the mechanism by which cardiomyocyte contraction is triggered is termed “excitation-contraction coupling.” Changes in intracellular Ca2+ concentration are central to this mechanism.

Without being limited by theory, PanLN and PanLN-forte are believed to bind to extracellular targets/receptors thereby activating a signal transduction cascade that ends with the cardiomyocyte shifting the Calcium-force curve.

In certain aspects, PanLN and PanLN-forte can be used to induce calcium sensitization, or sensitivity to calcium, in a myocardial cell. In still other aspects, PanLN and PanLN-forte can be used to treat a cardiovascular condition in a patient by inducing calcium sensitization, or a calcium sensitizing effect, in the patient.

In yet other aspects, in view of their ability to be imaged using near infrared fluorescence, PanLN and PanLN-forte can be used to study and identify signal pathways which further induce calcium sensitization in myocardial cells.

In still other aspects, the disclosure provides for a method for determining the signal transduction pathway associated with calcium sensitization in a myocardial cell comprising contacting a myocardial cell with PanLN or PanLN-forte and observing the expression of one or more markers or receptors associated with the presence or absence of contractile force changes of the cell. In certain embodiments, the method of determining the signal transduction pathway of the disclosure utilizes one or more markers or receptors associated with the presence or absence Ca2+ within the cell. In certain embodiments, the method of determining the signal transduction pathway can be achieved by measuring changes in protein phosporylation, changes in ion transport, changes in intracellular second messengers, or changes in other biochemical parameters before and after contacting the myocardial cell with PanLN or PanLN-forte. In other embodiments, the method of determining the signal transduction pathway can be measured by determining the activation of G-coupled-protein receptors, the activation of InsP3 receptors; or the activation of RyR receptors. In certain embodiments, the a method for determining the signal transduction pathway associated with calcium sensitization can be achieved (a) contacting a myocardial cell with PanLN or PanLN forte; (b) irradiating the myocardia cell at a wavelength absorbed by PanLN or PanLN forte; and (c) detecting a signal from PanLN or PanLN forte to identify the binding cite associated with PanLN forte.

In yet another aspect, the disclosure provides for a method of identifying a calcium sensitizer compound comprising (a) contacting a myocardial cell with PanLN or PanLN forte to bind the cell at a binding site associated with PanLN or PanLN forte; (b) performing a competitive assay or a displacement assay with one or more therapeutic agents to determine which therapeutic agents will bind to the binding site associated with PanLN or PanLN forte; and (c) optionally performing an assay to confirm the therapeutic agents identified in step (b) bind to the binding site associated with PanLN or PanLN forte. In other embodiments, the method further includes the steps or (a1) irradiating the myocardial cell at a wavelength absorbed by PanLN or PanLN forte and (a2) detecting a signal from PanLN or PanLN forte to identify the binding site associated with Pan LN or PanLN forte after the step of contacting the myocardial cell (a). In certain embodiments, the therapeutic agents identified in the method of the disclosure are then administered to a cell, subject, or patient in need thereof. In certain other embodiments, the therapeutic agents are small-molecule compounds, proteins, peptides, antibodies, or other therapeutic agents or combinations thereof.

### Compositions

Pharmaceutical compositions comprising combinations of an effective amount of at least one compound as described herein, and one or more of the compounds otherwise described herein, all in effective amounts, in combination with a pharmaceutically effective amount of a carrier, additive or excipient, represents a further aspect of the present disclosure.

The present disclosure includes, where applicable, the compositions comprising the pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of compounds according to the present disclosure in local ocular administration routes may also be used. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. Administration of compounds according to the present disclosure as eye drops, intravitreous injections, sub-tenon injections, and sub-retinal injections may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, intraocularly, to the ocular surface, rectally, nasally, buccally, vaginally or via an implanted reservoir. The term “parenteral” as used herein includes subcutaneous, intravenous, intramuscular, intra-articular, intra-synovial, intra-vitreous, sub-retinal, retinal, sun-tenon intrasternal, intrathecal, intrahepatic, intralesional and intracranial injection or infusion techniques. Preferably, the compositions are administered by local ocular administration, orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. In certain embodiments, pharmaceutical compositions for oral administration include formulations which aid in delivering the compound across the blood-retina barrier.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. In certain embodiments, pharmaceutical compositions for ophthalmic or local ocular use include lipophiliccally modified compositions and transplantable carriers.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition as described herein that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-20 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time. If the concentration of the active compound is too high, arrthymogenicity can occur. In certain embodiments, an effective amount of the compounds of the disclosure are sufficient to treat or ameliorate the condition being treated while avoiding arrthymogenicity.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, including epidermal growth factor receptor inhibitors, EPO and darbapoietin alfa, among others. In certain preferred aspects of the disclosure, one or more compounds according to the present disclosure are coadministered with another bioactive agent, or a wound healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Methods

In an additional aspect, the description provides therapeutic compositions comprising an effective amount of a compound as described herein or salt form thereof, and a pharmaceutically acceptable carrier. The therapeutic compositions can be used for treating or ameliorating cardiovascular disease states or conditions in a patient or subject, for example, an animal such as a human. The therapeutic compositions can be used for treating or ameliorating cardiovascular disorders or conditions in a patient or subject, for example, an animal such as a human. The therapeutic compositions can be used for treating or ameliorating narcolepsy or narcoleptic conditions in a patient or subject, for example, an animal such as a human. The therapeutic compositions can be used for treating or ameliorating asthma or asthmatic conditions in a patient or subject, for example, an animal such as a human. The therapeutic compositions can be used for treating or ameliorating cardiovascular disorders or conditions in a patient or subject, for example, an animal such as a human by inducing calcium sensitization in the subject

The terms “treat”, “treating”, and “treatment”, etc., as used herein, refer to any action providing a benefit to a patient for which the present compounds may be administered, including the treatment of an ophthalmic disease state or condition. The description provides therapeutic compositions as described herein for treating heart failure, heart disease, heart block, cardiac arrest from heart block, Bradycardia, Bronchospasm during anesthesia, cardiogenic shock, hypovolemic shock, Torsades de pointes, Beta-blocker overdose, ventricular arrhythmias, Short QT syndrome, Electrical storm in patients with Brugada syndrome, asthma, and narcolepsy.

In certain embodiments, the method comprises administering an effective amount of a compound as described herein, optionally including a pharmaceutically acceptable excipient, carrier, adjuvant, another bioactive agent or combination thereof.

In certain embodiments, the method comprises administering a compound as described herein in an amount effective to induce calcium sensitization in a subject, optionally including a pharmaceutically acceptable excipient, carrier, adjuvant, another bioactive agent or combination thereof.

In additional embodiments, the description provides methods for treating or ameliorating a cardiovascular disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In additional embodiments, the description provides methods for treating narcolepsy in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating a narcolepsy in the subject.

In additional embodiments, the description provides methods for treating asthma in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating asthma in the subject.

In additional embodiments, the description provides methods for inducing a calcium sensitizing effect in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or increasing sensitivity to calcium or inducing a calcium sensitizing effect in the subject.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered.

The term “pharmaceutically acceptable salt” is used throughout the specification to describe, where applicable, a salt form of one or more of the compounds described herein which are presented to increase the solubility of the compound in the gastric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### Kits

In an additional aspect, the description provides kits which, when used by the medical practitioner, can simplify the administration of appropriate amounts of the compounds of the disclosure or pharmaceutically acceptable salts, solvates or hydrate thereof to a patient or cell.

A typical kit of the disclosure comprises one or more units dosage forms of a compound of the disclosure or pharmaceutically acceptable salts, solvates or hydrates thereof, and instructions for administration to a subject or cell. A typical kit of the disclosure could also, or alternatively, contain a bulk amount of a compound of the disclosure or pharmaceutically acceptable salts, solvates or hydrates thereof.

Kits of the disclosure can further comprise devices that are used to administer a compounds of the disclosure inhibitor or pharmaceutically acceptable salts, solvates or hydrates thereof, and instructions for administration to a subject or cell. Examples of such devices include, but are not limited to, intravenous cannulation devices, syringes, drip bags, patches, topical gels, pumps, containers that provide protection from photodegradation, autoinjectors, and inhalers.

Kits of the disclosure can further comprise pharmaceutically acceptable vehicles that can be used to administer one or more compounds of the disclosure as active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

The examples presented in this disclosure are put forth so as to provide those of ordinary skill in the art with a complete disclosure and description of how to make and use the compounds, compositions, and therapeutic methods of the invention, and are not intended to limit the scope of what the inventors regard as their invention.

### Example 1: Pharmacological Review of PanLN

The pharmacological activity of PanLN (trifluoroacetate salt) was evaluated in 168 biochemical assays. Biochemical assay results are presented as the percent inhibition of specific binding or activity throughout the report. All other results are expressed in terms of that assay's quantitation method.

Significant responses (≥50% inhibition or stimulation for Biochemical assays) were noted in the primary assays listed in the tables below:

| Assay Name | Species | Conc. | % Inh. |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

indicates data missing or illegible when filed

| Assay Name | Species | Conc. | % Inh. |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

indicates data missing or illegible when filed

### Example 2: Adult Human Primary Ventricular Cardiomyocyte Contractility Assay with PanLN Triflutate

This study was aimed at quantifying the effects of PanLN Triflutate on contractility transients generated in adult human primary ventricular cardiomyocytes dissociated from human donor hearts to determine potential risks related to contractility and arrhythmia.

All human tissues used for the study were obtained by legal consent from organ donors in the US. Donor hearts from males and females were harvested using proprietary surgical techniques and tools and were shipped to AnaBios via dedicated couriers. Upon arriving, each heart was assigned a unique identifier number that was reproduced on all relevant medical history files, data entry forms and electronic records.

### Table 2: Experimental Parameters

| Parameter | Value |
|---|---|
| Tissue type | Adult Human Primary Ventricular |
| | Myocytes |
| Temperature | Approx. 35° C. |
| Perfusion mode | Continuous perfusion |
| Flow rate | Approx. 2 mL/min |
| Acquisition rate | 150 Hz |
| Stimulation rate | 1.0 Hz |
| Stimulation intensity | 1.5x threshold |
| Equilibration time | 5 min or steady state |
| Recording Buffer | Buffer Solution + 0.1% DMSO |
| | (See Buffer composition) |
| Positive Control | 200 nM Dofetilide |
| | (not reported) |
| Assay format: Number | Minimum of 5 cells × 4 |
| cells & concentrations | concentrations |
| Vehicle/Baseline control | Buffer solution + 0.1% DMSO |

TABLE 3
Test Article, Postive Control And Vehicle/Control Article Formulation

| Test Article Identity | | | Stock Solution |
|---|---|---|---|
| MW | Concentrations Tested | Storage | Formulation and Storage |
| PanLN Triflutate | 0.2, 1, 5, 25 μM | Powder stored at −20° C. and | 250 μM stock in recording |
| MW: 788 (da) g/mol | | protected from light. | buffer will be prepared fresh |
| | | | on day of experiment. |
| | | | Remainder will be protected |
| | | | from light and discarded |
| | | | after the experiments are |
| | | | finished. |
| Positive control | 200 nM | Powder stored at −20° C. | 2 mM stock in 100% DMSO. |
| Dofetilide | | Desiccated, protected from | Prepared fresh daily. |
| MW: 441.6 g/mol | | light. | Protected from light. |
| Vehicle/Baseline | Recording Buffer + | 4° C. | N/A |
| | 0.1% DMSO | | |

TABLE 4
Recording Buffer Composition

| | | |
|---|---|---|
| NaCl | 145 | mM |
| KV | 4.0 | mM |
| MgCl₂ | 1.0 | mM |
| Dextrose | 11.1 | mM |
| HEPES | 10.0 | mM |
| CaCl₂ | 1.8 | mM |

TABLE 5
Stimulation Protocol And Test Article Application Sequence

| Perfusion | Vehicle | | | | | |
|---|---|---|---|---|---|---|
| Sequence | Control | 0.2 μM | 1 μM | 5 μM | 25 μM | Wash |
| Treatment | 120 sec | 300 sec | 300 sec | 300 sec | 300 sec | 300 sec |
| Time & | 1.0 Hz | 1.0 Hz | 1.0 Hz | 1.0 Hz | 1.0 Hz | 1.0 Hz |
| Stimulation | | | | | | |

### Experimental Procedure

### Isolation of Adult Human Primary Ventricular Myocytes

1. Adult human primary ventricular myocytes were isolated from ethically consented donor hearts. A proprietary protocol was employed to enzymatically digest hearts and isolate cardiomyocytes.

### Contractility Recordings Procedures

1. Cardiomyocytes were placed in a perfusion chamber mounted on the stage of an inverted Olympus IX83P1ZF microscopes.
2. Cardiomyocytes were then continuously perfused at approximately 2 ml/min with recording buffer heated to 35±1° C. using an in-line heater from Warner Instruments.
3. Cardiomyocytes were equilibrated for 5 minutes under constant perfusion.
4. The cells were stimulated with supra-threshold voltage at a 1 Hz pacing frequency (bipolar pulse of 3 ms duration) on a field stimulator with a pair of platinum wires placed on opposite sides of the chamber connected to a MyoPacer stimulator.
5. Starting at 1 V, the amplitude of the stimulating pulse was increased until the cardiomyocytes started generating contraction-relaxation cycles, and a value 1.5× threshold was used throughout the experiment.
6. Cardiomyocytes were then imaged 150 Hz using an Optronis Cyclone-25-150-M camera. Digitized images were displayed within the MyoBLAZER acquisition software.
7. The longitudinal axis of the selected cardiomyocytes was aligned parallel to the video raster line, by means of a cell framing adapter.
8. Optical intensity data was collected from a user-defined rectangular region placed over the cardiomyocyte image. The optical intensity data represented the bright and dark bands corresponding to the Z-lines of the cardiomyocyte. The MyoBLAZER Analysis software analyzed the periodicity in the optical density of these bands by means of a fast Fourier transform algorithm.

### Analyses of Inotropy and Pro-Arrhythmia Markers

1. Contractility Amplitude (CA) was calculated as the peak minus baseline of the contractility transient 2. Stability was assessed by continuous recording for 2 minutes in baseline control conditions.
3. Each test article concentration was applied for a period of 300 seconds.
4. At the end of the cumulative test article additions, a 5 min washout period was implemented.
5. An application of 200 nM Dofetilide was applied to a separate group of cells as a positive control for pro-arrhythmic events.
6. Contractility Amplitude values were calculated from the average of the last 20 contractility transients
7. The averaged CA were expressed relative to each cardiomyocyte's specific baseline control condition and graphed in concentration-response plots. Results were expressed as mean±SEM.
8. Dose response curves were fitted to the Hill equation using SigmaPlot to determine IC50 or EC50 values.
9. Aftercontractions (AC) were identified as spontaneous secondary contraction transients of the cardiomyocyte that occurred before the next regular contraction and that produced an abnormal and unsynchronized contraction.
10. Contraction Failure (CF) was identified when an electrical stimulus was unable to induce a contraction.
11. Alternans and Short-Term Variability (STV) are visualized in Poincare plots of Contraction Amplitude variability. STV was calculated with the last 20 transients of each control and test article concentration period. Alternans were identified as repetitive alternating short and long contractility amplitude transients. STV values were normalized to the vehicle control value of each cell.
Mean±SEM
% Incidence
Short-term variability analysis

$$STV = Σ \vert Can + 1 - CAn \vert {( 20 \times √ 2 )}^{- 1}$$

12. AC, CF and Alternans were plotted and expressed as % of incidence of cells exhibiting each of the signals.

### Evaluation Parameters

1. Contractility
   a. Contractility Amplitude (CA) (%)
2. Pro-Arrhythmia
   a. After-Contraction (AC) (%)
   b. Contractility Failure (CF) (%)
   c. Alternans (%)
   d. Short Term Variability of CA (STV (CA))

### Contractility Analysis

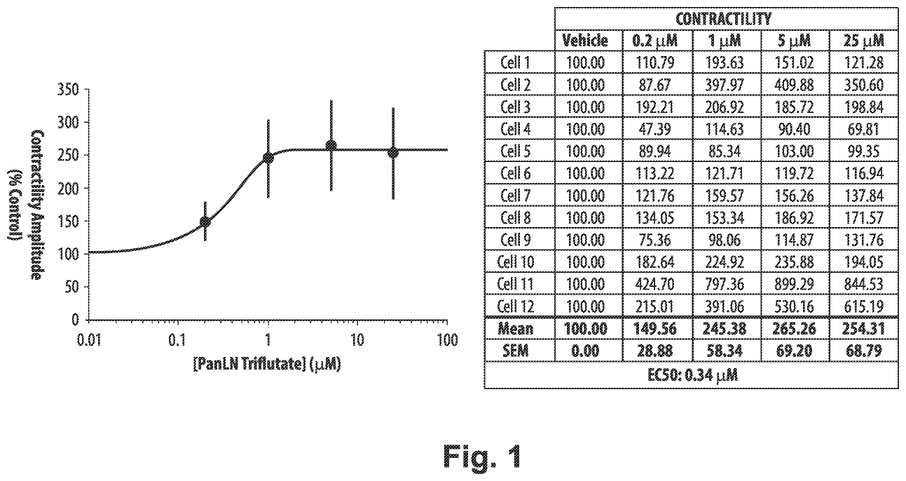

Percent Changes in Contractility Amplitude for PanLN Triflutate at 1 Hz. (n=12) (See, FIG. 1)

### Pro-Arrhythmia Analysis

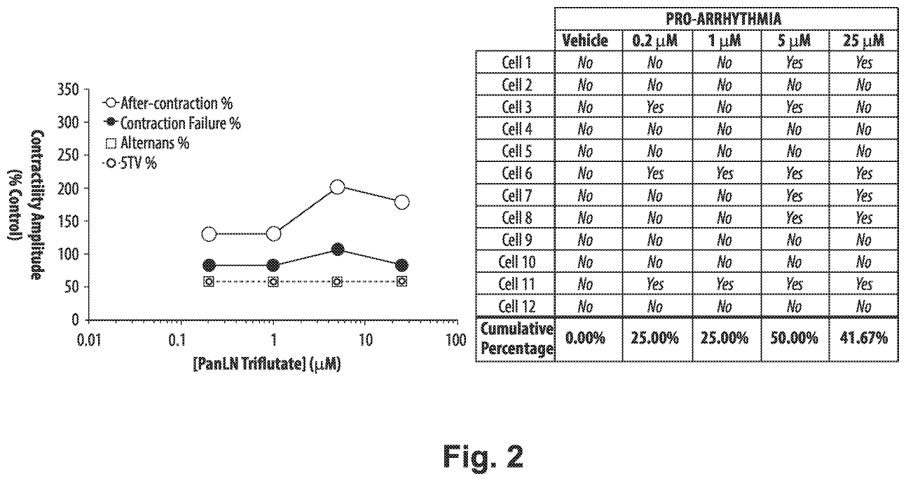

Percent Incidence in AC, CF, Alternans and STV for PanLN Triflutate. (n=12) (See FIG. 2)

Several episodes of arrhythmias, with contraction failure, lasted for longer than 15-30 seconds were observed in 3 cells: Cell 1 at 25 μM; Cell 3 at 0.2 μM and 1 μM; Cell 7 at 1 μM and 5 μM. Two typical examples are presented below.

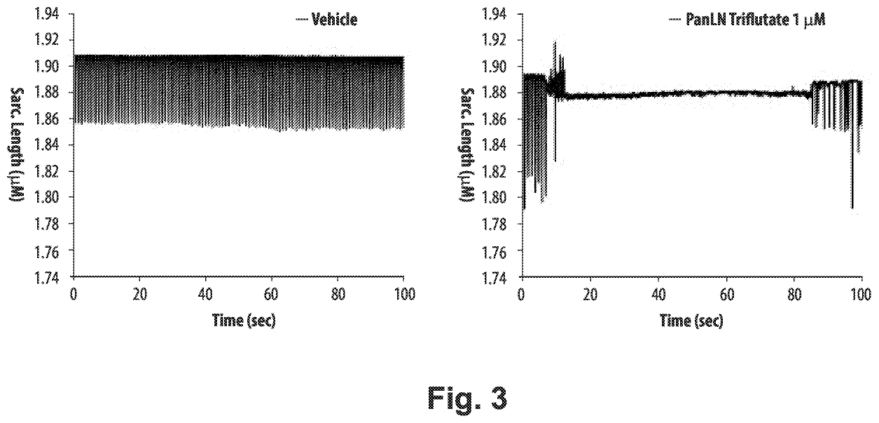

Typical contractility transients recorded from a ventricular cardiomyocyte at a pacing of 1 Hz in the presence of vehicle control and after exposure to 1 μM PanLN Triflutate. (See, FIG. 3).

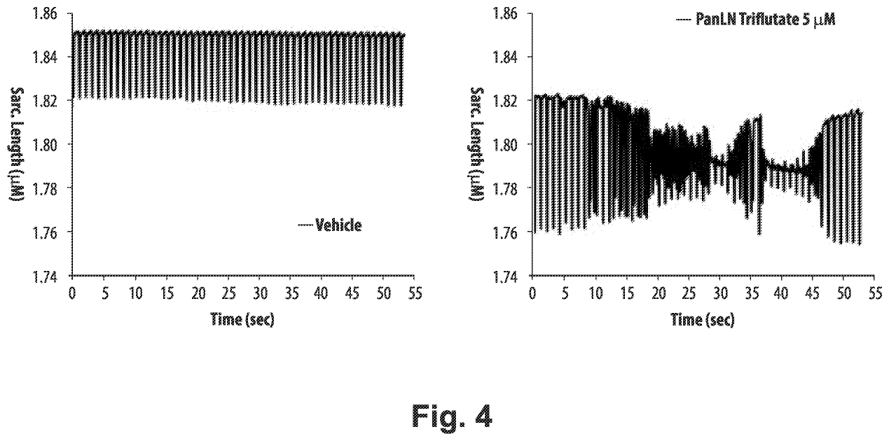

Typical contractility transients recorded from a ventricular cardiomyocyte at a pacing of 1 Hz in the presence of vehicle control and after exposure to 5 μM PanLN Triflutate. (See, FIG. 4).

### CONCLUSIONS

PanLN Triflutate increased contractility amplitude with an EC50 value of 0.34 μM. Moreover, PanLN Triflutate demonstrated a pro-arrhythmic potential and caused several episodes of arrhythmia.

### INCORPORATION BY REFERENCE

The entire contents of all patents, published patent applications and other references cited herein are hereby expressly incorporated herein in their entireties by reference.

### EQUIVALENTS

While the present disclosure has been described with respect to a number of embodiments and examples, those skilled in the art, having benefit of this disclosure, will appreciate that other embodiments can be devised which do not depart from the scope and spirit of the present disclosure.

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired products, additional ingredients may be added, and/or similar ingredients may be substituted for one or more of the ingredients described.

Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein.

1. A pharmaceutical composition comprising 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof and a pharmaceutically acceptable excipient or carrier.

2. A pharmaceutical composition comprising 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium or a salt or hydrate thereof and a pharmaceutically acceptable excipient or carrier.

3. A method of treating a cardiovascular disease, asthma, or narcolepsy in a patient in need thereof comprising administering a pharmaceutically effective amount of 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof.

4. The method of claim 3, wherein the cardiovascular disease is heart failure, heart disease, heart block, cardiac arrest from heart block, bradycardia, cardiogenic shock, hypovolemic shock, Torsades de pointes, ventricular arrhythmia, Short QT syndrome, or electrical storms associated with Brugada syndrome.

5. The method of claim 3, wherein the compound is administered in the form of a pharmaceutical composition wherein the pharmaceutical composition comprises the compound and one or more pharmaceutically acceptable excipients or carriers.

6. (canceled)

7. A method of treating a cardiovascular disease in a patient in need thereof comprising administering 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce calcium sensitization in the patient.

8. The method of treating a cardiovascular disease in a patient according to claim 7 wherein the cardiovascular disease is heart failure; cardiogenic shock; septic shock; pulmonary hypertension; bradycardia; high blood pressure, angina, arrhythmia hypertrophic obstructive cardiomyopathy or an allergic reaction.

9. A method of inducing a calcium sensitizing effect in a myocardial cell comprising administering 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce a calcium sensitizing effect in the myocardial cell.

10. A method of inducing a calcium sensitizing effect in a patient in need thereof comprising administering 2-((E)-2-((E)-2-(4-(2-carboxyethyl)phenyl)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, 2-((E)-2-((E)-2-(4-(2-carboxyethyl)phenoxy)-3-((E)-2-(3,3-dimethyl-1-(3-(trimethylammonio)propyl)indolin-2-ylidene)ethylidene)cyclohex-1-enyl)vinyl)-3,3-dimethyl-1-(3-(trimethylammonio)propyl)-3H-indolium, or a salt or hydrate thereof in an amount effective to induce a calcium sensitizing effect in the patient.

11-18. (canceled)
